(19) United States
(12) Patent Application Publication
(10) Pub. No.: US 2005/0113931 A1
(43) Pub. Date: May 26, 2005

(54) Endoprosthesis for replacing a joint, especially a shoulder joint

(76) Inventor: Willi Horber, Zurich (CH)

Correspondence Address: BURNS DOANE SWECKER & MATHIS L L P, POST OFFICE BOX 1404, ALEXANDRIA, VA, 22313-1404, US

(21) Appl. No.: 10/984,831

(22) Filed: Nov. 10, 2004

Related U.S. Application Data
(63) Continuation of application No. PCT/CH03/00295, filed on May 7, 2003.

(30) Foreign Application Priority Data
May 15, 2002 (CH) 825/02

(51) Int. Cl. A61F 2/40; A61F 2/30

(52) U.S. Cl. 623/19.14; 623/22.46; 623/23.47

(57) ABSTRACT

A joint prosthesis is disclosed which includes an articulation cavity for an articulation body, in a head section of a base part. The position of the articulation body used in the articulation cavity defines the subsequent collar axis for an articulation part mounted on the articulation body. The articulation cavity has a tapering lateral surface which co-operates with an articulation surface that is symmetrical to a pivoting axis. The articulation surface has a circular edge. In an cutting plane perpendicular to the pivoting axis, which passes through the contact point between lateral surface and the circular edge, the radius of the circular edge is larger than the radius at the vertex circle of the cutting curve between the cutting plane and the lateral surface. Two contact points can thus be created between the circular edge and the lateral surface, said contact points being located on different sides of a plane containing the rotation axis and the pivoting axis. In this way, a rolling movement of the articulation body in the articulation cavity can be prevented.

### CROSS REFERENCE TO RELATED APPLICATION

This application claims priority under 35 U.S.C. §119 to Swiss Application CH 2002 825/02 filed in Switzerland on May 15, 2002, and as a continuation application under 35 U.S.C. §120 to PCT/CH03/00295 filed as an International Application on May 7, 2003, designating the U.S., the entire contents of which are hereby incorporated by reference.

### FIELD OF THE INVENTION

The invention relates to an endoprosthesis for replacing a joint, such as a shoulder joint. Such an endoprosthesis includes a base part in which an articulation cavity, tapering towards the bottom, is provided with an axially symmetrical lateral surface. The rotation axis of this lateral surface is positioned in the direction of the joint neck. In the articulation cavity an articulation body is arranged, which interacts at some contact points with the lateral surface by an articulation surface axially symmetrical to a pivoting axis. The pivoting axis of this articulation cuts the rotation axis. Accordingly the articulation body is hinged to the base part rotatable around the rotation axis of the lateral surface and around the pivoting axis. Therefore, by the articulation body supporting a first articulation part, the first articulation part is alignable with respect to its position of inclination and rotation relative to the base part and, in the chosen alignment, fixable within the articulation cavity by a locking means. The artificial first articulation part is provided for the articulation with a natural or with an artificial second articulation part.

### DESCRIPTION OF THE RELATED ART

An endoprosthesis for a shoulder joint is known from DE-299 18 589 U1, in which an articulation cavity is formed in a base part. The articulation cavity is formed as a cone. A rotating piece is arranged in this articulation cavity. The rotating piece is rotatable around the axis of the cone and connectable to the base part in any rotation position by means of a cone deadlock. A directional piece is hinged to the rotating piece. The directional piece supports a cap and is pivotable with respect to the rotating piece around a cylinder axis cutting the collar axis perpendicularly. This endoprosthesis enables the directional piece to be articulated at the base part not on spherical surfaces but on the surfaces of axial rotation bodies. By that it is possible to adjust the rotation position and the inclination position of the cap independently from each other. The endoprosthesis also includes a locking means for the fixing of the articulation body in the articulation cavity.

### BACKGROUND OF THE INVENTION

The international patent application WO02/39933 (PCT/CH01/00676) describes an endoprosthesis for a shoulder joint in which an axially symmetrical articulation space is formed within the head section of the base part. In this articulation space an axial articulation body is articulated. The axis of the articulation body stands perpendicular to the rotation axis of the articulation space. In an example the articulation space has a conical base. This base serves as a clamping surface, co-operating with an axially symmetrical articulation surface of the articulation body. In order to obtain a good interlock of the articulation surface to the clamping surface it is proposed to form these surfaces by a number of edges. In this endoprosthesis it is achieved that, with easy-to-build axially symmetrical surfaces, both the inclination and the rotation position of a cap connected to the articulation body can be obtained. As compared to the subject of DE-299 18 589 U1, the subject of WO02/39933 does not need a rotation piece. The embodiments of WO02/39933 show a spectrum of possible variations, which at least partly are applicable also to this invention. The content of WO02/39933 is hereby incorporated by reference in its entirety.

In the embodiments described in WO02/39933 the articulation body shows a linear arrangement of the contact points between the articulation body and the base part. For this reason when the locking means is loose the articulation body, under the influence of a respective force, tends to roll with the articulation surface on the clamping surface.

### SUMMARY OF THE INVENTION

An endoprosthesis is disclosed for the replacement of a joint, in particular a shoulder joint, in which an axial articulation body is fixable within an axially symmetrical articulation cavity in any position desired in terms of inclination and rotation and in which an unintentional rolling movement between the articulation body and the articulation cavity is prevented. In an advantageous exemplary embodiment, a clamping action is provided between the articulation body and the articulation cavity.

### BRIEF DESCRIPTION OF THE DRAWING FIGURES

The figures illustrate exemplary embodiments of shoulder prostheses, wherein:

FIG. 1 shows a view of the head section, the articulation body and the locking screw according to a first exemplary embodiment;

FIG. 2 shows a side view of the articulation body from FIG. 1;

FIG. 3 shows a top view of the articulation body from FIGS. 1 and 2;

FIG. 4 shows a top view of the head section with the articulation cavity;

FIG. 5 shows a top view of the head section with the inserted articulation body;

FIG. 6 shows a view of an entire exemplary endoprosthesis without cap and without glenoid;

FIG. 7 shows a vertical section through the endoprosthesis according to FIG. 6;

FIG. 8 shows a section according to FIG. 7, but with cap and glenoid;

FIG. 9 shows an exemplary schematic section normal to the pivoting axis of the articulation surface and cutting two contact points of the articulation surface with the lateral surface of the articulation cavity;

FIG. 10 shows a section through double-stepped articulation cavity 22 according to a second exemplary embodiment with an articulation body articulated in it with four circular edges;

FIG. 11 shows a schematic illustration of an exemplary arrangement of contact points between articulation cavity and articulation body;

FIG. 12 shows a view from below the articulation body according to FIG. 10;

FIG. 13 shows a top view of the articulation body according to FIGS. 10 and 12;

FIG. 14 shows an exemplary geometric illustration of a geometry, that guarantees two contact points between one circular edge or the articulation surface and the lateral surface;

FIG. 15 shows an exemplary embodiment with a three-point seating between articulation body 15 and lateral surface 25; and

FIG. 16 shows the embodiment according to FIG. 15, with an inserted articulation body 15 rotated at 180 degrees.

### DETAILED DESCRIPTION

In an exemplary embodiment, an articulation surface interacts with the lateral surface at at least three contact points separated one from the other. The articulation surface includes at least one axially symmetrical circular edge around the pivoting axis, which interacts with the lateral surface in two contact points distant one from the other. Both contact points of the circular edge are arranged, with respect to a plane lodging the pivoting axis and the rotation axis, in such a way that a contact point lies on one side and the other contact point lies on the other side of this plane. A geometrically linear or punctual contact is impossible in practice. Physically spoken contact points will always be present as surface areas. Therefore, linear edges are also considered as articulation surfaces.

The radius of the circular edge is larger than the radius of a vertex circle of a cutting curve between a conical sheath surface and a plane perpendicular to the pivoting axis cutting the contact points. The cutting curve is an hyperbole. The conical sheath surface is created through the rotation of a straight line around the rotation axis. This straight line cuts the rotation axis and touches a contact point. This straight line belongs to a tangential plane defined at the contact point by a first and a second tangent. The first tangent lies in the plane perpendicular to the pivoting axis and runs tangent to the circular edge at the contact point. The second tangent lies in the plane perpendicular to the rotation axis and runs tangent to the lateral surface at the contact point.

In an advantageous exemplary embodiment, the articulation surface includes, or alternately consists of, at least two axially symmetrical circular edges round the pivoting axis, at least one of which shares two contact points, distant one from the other, with the lateral surface. One of these contact points is lying on one side of a plane lodging the pivoting axis and the rotation axis, whereas the other contact point is lying at the other side of this plane.

The lateral surface can be preferably formed conically. Therefore, it can have a conical shape with straight lateral surface lines, a trumpet shape with lateral surface lines convex towards the rotation axis or an egg holder shape with lateral surface lines concave towards the rotation axis. In case of a conical shape, the circular edge, at the contact points between the circular edge and the lateral surface, can have a radius, with respect to the pivoting axis, that is larger than the vertex radius of an cutting curve between the lateral surface and a plane perpendicular to the pivoting axis cutting the contact points. In both cases with bent lateral surface lines the radius of the circular edge can be formed larger than the vertex radius of a cutting curve between the plane perpendicular to the pivoting axis cutting the contact points and a conical sheath surface touching the lateral surface. This conical sheath surface is created by the rotation round the rotation axis of a tangent to the conical sheath line at the contact point. By that it is achieved that the articulation body does not co-operate only linearly with a lateral surface but has at least a three-point support. This arrangement, through the appropriate choice of the lateral surface and of the articulation surface, allows a clamping action to be achieved between the articulation cavity and the articulation body.

If two or more circular edges meet the above mentioned criteria, two or more contact points lie on one side and two or more contact points lie on the other side of a plane lodging the rotation axis and the pivoting axis. By this a quadrangular arrangement of the contact points is obtained. This non-linear arrangement of the contact points can prevent, together with the three-point support, the rolling of the articulation body on the lateral surface. The radiuses of the circular edges can be different. In circular edges with the same radius the pivoting axis usually cuts the rotation axis perpendicularly.

In an exemplary embodiment, both circular edges distanced one from the other do not lie on a plane parallel to the rotation axis. The articulation body can, to a limited degree, also spin transversally to the pivoting axis. This results in a certain lateral displacement (offset) of the articulation body with respect to the rotation axis. This offset can be exploited in the orientation of the articulation body, for example to optimize the position of the axis of the joint neck and of the articulation part, respectively, placed on the articulation body in a shoulder joint.

The lateral surface of the articulation cavity can be structured in the shape of a trumpet, of an egg holder or of a cone. The lateral surface can be formed by a surface or by one or more ring edges. The ring edge can be annular or threaded. If the lateral surface has at least one ring edge, the circular edge at the contact points between the circular edge and the ring edge preferably has a radius, with respect to the pivoting axis, which is larger than the vertex radius of a cutting curve between the plane perpendicular to the pivoting axis cutting the contact points of a circular edge and a conical sheath surface. For the construction of this conical sheath surface a plane including the tangent to the ring edge and the tangent to the circular edge at the contact point can be cut through with a rotation axis. A straight line through the cut point obtained on the rotation axis and the contact point can be made to rotate around the rotation axis. The resulting conical sheath surface is now cut at the contact points by a plane perpendicular to the pivoting axis. If the vertex radius of this cutting curve is smaller than the radius of the circular edge, the circular edge adheres to the ring edge in two points.

In the case of a ring edge no offset occurs when the articulation body is pivoted transversally to the pivoting axis. This can be exploited by combining a conical lateral surface with a ring edge to exclude the possibility of an offset. With a lateral surface combined in this way, the articulation body can be configured to lie on all possible contact points only when the pivoting axis stands in a preset angle, usually perpendicularly to the rotation axis. By tightening the locking means the articulation body is brought into this position.

In an advantageous embodiment the angle between the actual lateral surface or the constructed conical sheath surface and the rotation axis at the contact points lies in an area from 0 to 30°, preferably between 2 and 20°, and especially preferred between 5 and 15°. By this a clamping action is obtained between the articulation surface and the lateral surface. With an angle between 0 and 5 degrees the clamping action is at its highest, while within this range of the angle the plunging depth has greater tolerances. With angles greater than 15 degrees the clamping action is reduced while the plunging depth can be determined more precisely in advance. Larger angles of less than 90 degrees are also possible if the clamping action is given up. The clamping action enables the articulation body to be provisionally fixed in the articulation cavity by a light pressure. By this the position of the collar axis can be controlled before this position is fixed by tightening the locking means and therefore, before the articulation surface or the lateral surface is deformed.

Thanks to the relatively high pressure forces generated at the contact points when tightening the locking means, at least one articulation surface and lateral surface are deformed. Depending on the material pairing it can be obtained that the articulation surface digs into the lateral surface at the contact points or that the articulation surface, i.e. the circular edges of the articulation body is deformed. In the last case the lateral surface flattens the circular edges at the contact points. Thanks to these deformations at the contact points, the pivoting and the rotation of the articulation body around the pivoting axis and around the rotation axis, respectively, are safely prevented. The hardness of the material used for the articulation body, the locking means and the articulation cavity can therefore be chosen according to the intended deformation. Also when these parts can be produced with the same material or with a material of the same hardness, a different pairing can be advantageous. It is particularly advantageous to have the articulation body made of a material that is softer than the articulation cavity and the locking means.

Another possibility to increase the clamping stability is the structuring of the circular edges and/or lateral surface with protruding and sharp edges or points, with notches, grooves or grid relieves. In this case it is advantageous to match the structuring of both surfaces with one another so that they bite during tight clamping.

The articulation body can have a central borehole with an axially symmetrical base. The rotation center or the rotation axis of the base can coincide with the pivoting axis. At the base of the borehole in the articulation body an opening is provided through which a locking means runs. The opening is especially an oblong hole with oblong extension perpendicular to the pivoting axis. The locking means can, for example, be a screw connecting the articulation body to the base part. Other locking means are also possible such as expansion bolts or rivets. The base can be cylindrical, nipple shaped or spindle shaped. A spherical base is preferred due to manufacturing, technical and mechanical reasons. This axially symmetrical structure of the base allows the tightening of a screw head with a circular edge against the base. In this way the circular edge digs into the base. If the base is cylindrical with a cylinder axis coinciding with the pivoting axis of the articulation body the screw head digs on two points. These points lie on a plane transversal to the pivoting axis and at a distance from the pivoting axis. In this way a pivoting movement around the pivoting axis or the articulation body can be counteracted.

For additional safety of the clamping, adequate tools can be used such as pressing lids, counter screws or nuts, plastic or elastic deformable inserts between screw head and base or in the screw threads of the union nut.

Within certain limits the described geometry also allows the swiveling of the pivoting axis around a third axis normal to the pivoting axis and the rotation axis. A limit to this swiveling is created by the angle of the lateral surface at the contact points. As soon as the cutting curve between the plane normal to the pivoting axis through the contact points and the above defined conical sheath surface creates a parabola, a safe clamping between the articulation body and the articulation cavity can no longer be obtained. However, as long as the cutting curve is an hyperbole the articulation body can be safely fixed in the articulation cavity. In these circumstances, as described above, an offset is obtained. For this offset to be exploited, the opening must have a diameter greater than the diameter of the locking screw.

In place of or in addition to a securing through a borehole in the articulation body also other locking media can be used such as screw lids, pressing plates, guiding nuts and the like, as described in the examples of WO02/39933. The securing at the base of a borehole in the articulation body has the advantage that the articulation body can have a very compact structure. This allows a small cross-section area of the articulation body as well as a small diameter of the articulation cavity. This results in a small external diameter of the head section in which the articulation cavity is arranged. To further reduce the external diameter the opening margin of the articulation cavity features a reinforcing rib surrounding the opening margin. In this way the wall thickness of the head section can be minimal without running the risk of the wall collapsing or tearing under stress.

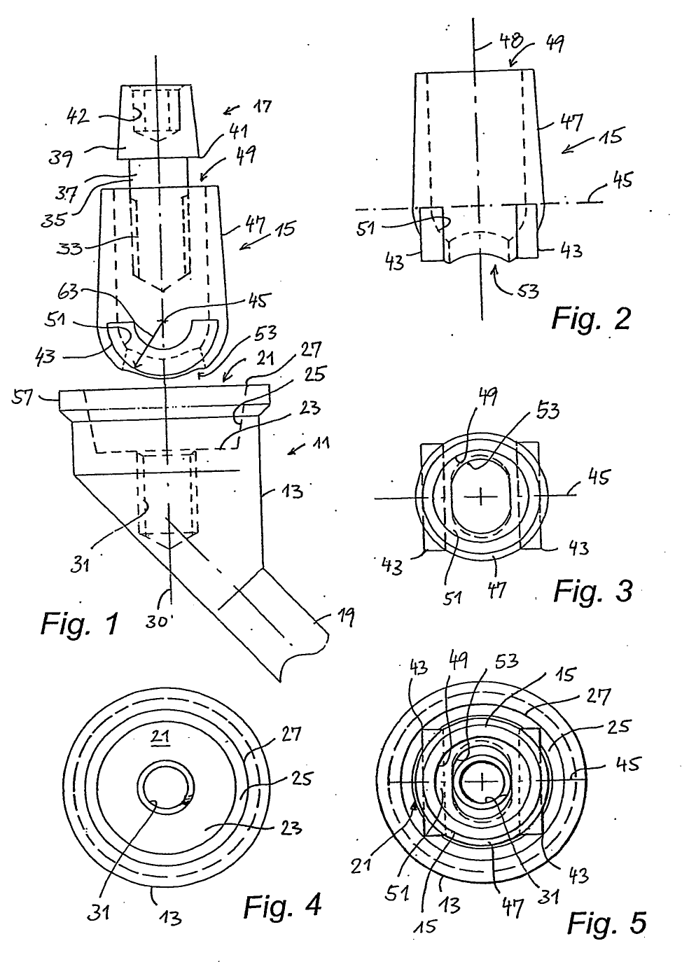

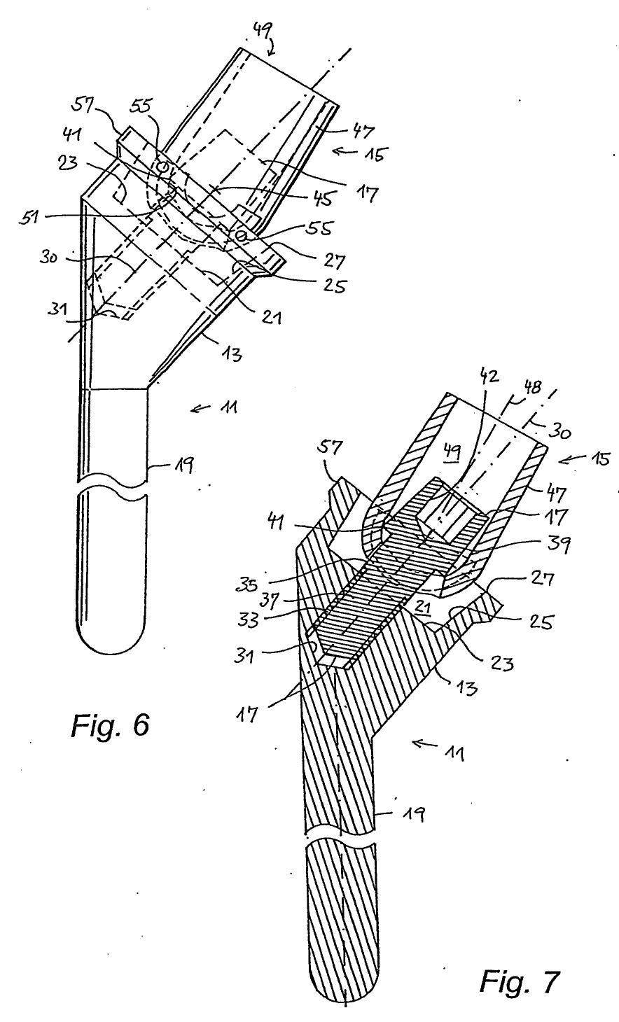

FIG. 1 shows a base part 11, an articulation body 15 and a locking screw 17 separated from each other. FIGS. 6 and 7 show the same parts assembled together. The base part 11 for insertion into the diaphysis, or metaphysis of the humerus, has a stem section 19 and a head section 13. In the head section 13 of the base part there is formed an articulation cavity 21. The articulation cavity 21 is structured as a truncated cone. It has a flat cavity base 23 and a conical lateral surface 25 as cavity wall. At its opening edge 27 the articulation cavity 21 has a diameter that is greater than the diameter at the cavity base 23. The angle between the conical lateral surface 25 and the rotation axis 30 is 10 degrees. The preferred angles are between 5 and 15 degrees, although greater or smaller angles are also possible. In the cavity base 23 at the rotation axis 30 there is a threaded hole 31. The dimension and thread of the threaded hole 31 match with the locking screw 17. The locking screw has a shaft 37 with a lower threaded section 33 and an upper shaft area 35 without threads. The locking screw 17 also includes a screw head 39. The screw head 39 has a tooth tip 41 with a larger radius than the radius of shaft 37. A contact surface 42 adjusted to a tool for the tightening of the locking screw 17 is available at the screw head 39. Between screw head 39 and articulation cavity 21, in particular between the tooth tip 41 and the conical lateral surface 25, the articulation body 15 can be clamped.

The articulation body 15 fits into the articulation cavity 21. The articulation body 15 has articulation surfaces 43 which are structured axially symmetrical to the pivoting axis 45. In this example the articulation surfaces are two circular edges 43. This example is not a limitative one. The articulation surfaces 43 can be continuous or interrupted. Interruptions in the articulation surface are obtained by means of cuts or slots in the surface or edge. Therefore, the articulation surface can also be made of a line of tips. Departing from the pure cone shape the articulation cavity 21 can be designed according to a trumpet shape or egg holder shape. In this case the lateral surface lines are not straight like in a cone but are convexly or concavely bent. The lateral surface can also feature circular grooves, threaded grooves or lateral surface line grooves. Lateral surface line grooves are preferably structured in such a way that the wall of the grooves adheres to the front side and the lateral side of the articulation body 15. This favors the tightening of the articulation body in the articulation cavity 21. The material of the lateral surface can be softer than the material of the articulation body.

The lateral surface can be molded with a three-dimensional grid relief. The lateral surface can be built with three or more cylindrical and concentric holes with gradually reducing diameters. In this way, when pressing the articulation body 15 into the holes, the orifice edges are cut in the articulation surfaces 43 of the articulation body 15. The articulation cavity 21 can have two or more conical lateral surfaces 25 in a gradually terraced manner. These can act with the same or with always different articulation surfaces 43 of the articulation body 15.

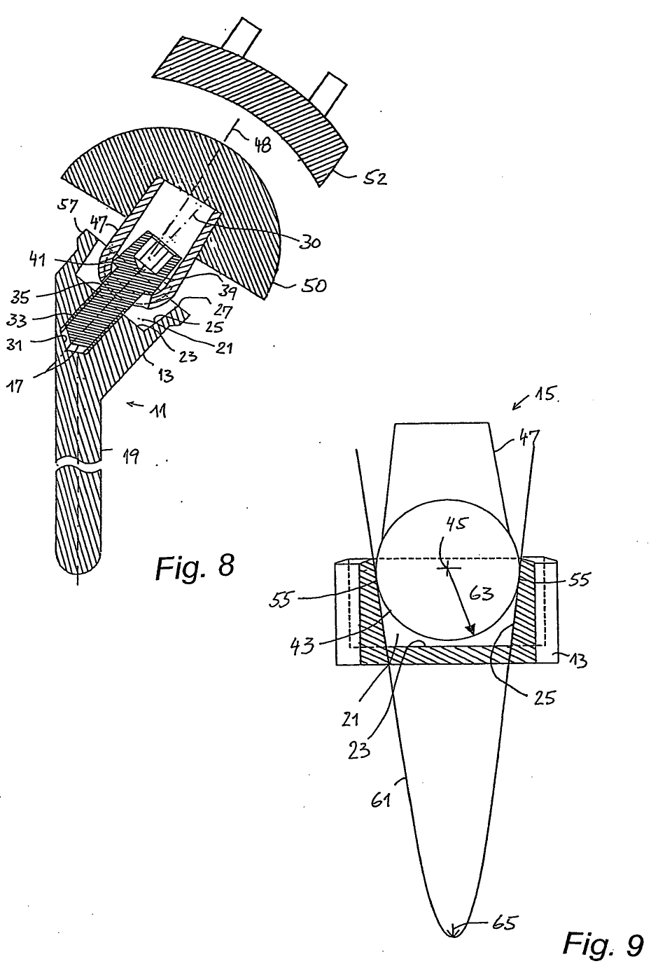

The articulation body 15 has an external conical surface 47 concentric to a collar axis 48 (FIG. 2). This conical surface 47 together with the collar axis 48 can be directed in a direction different from the direction of the rotation axis 30. On this conical surface 47 a cap 50 (FIG. 8) can be placed interacting with a natural or artificial glenoid 52 (FIG. 8). In the articulation body 15 and concentric to the conical surface 47 there is a bore 49. This bore 49 is essentially a blind bore with a spherical base 51. The base 51 can also be cylindrical or approximate to a hollow spherical surface. In the base 51 there is a slotted hole 53. Its width corresponds at least to the diameter of the shaft 37 of the locking screw 17. The length of the slotted hole 53 is extending in the direction of a pivoting around the pivoting axis 45 and defines the extent of the pivoting capacity of the articulation body. In this example, as can be seen from FIGS. 6 and 7, the pivoting capacity with respect to the rotation axis 30 is of about 10 degrees in both directions.

FIGS. 4 and 5 show a top view of the articulation cavity 21. In FIG. 4 the articulation cavity is empty. In FIG. 5 the articulation body 15 is inserted into the articulation cavity. FIG. 5 clearly shows how the articulation surfaces 43 touch the conical lateral surface 25 in four points. These points practically lie at the angular points of a rectangle, which in this example has sides of almost the same length. In FIG. 6 the contact points 55 are highlighted with circles. The cutting curve between the plane running perpendicular to the pivoting axis and passing through the contact points 25 and the conical lateral surface is a hyperbole 61 (FIG. 9). The vertex radius 65 of this hyperbole 61 is smaller than the radius 63 of the articulation surface 43. The contact points 55 lie, with increasing insertion depth, at a greater distance from the opening margin 27 of the articulation cavity 21. The deeper the insertion depth, the closer the contact points 55 move to the rotation axis 30.

The compression force needed for a long lasting clamping and interlock of the articulation body and articulation cavity is obtained by means of the screw connection. The articulation body 15 is clamped between the tooth tip 41 and the conical lateral surface 25. In order to prevent the force generated at the four contact points 55 from causing a deformation of the head section or leading to a tear in the wall of the articulation cavity 21, the orifice margin 27 of the articulation cavity 21 is reinforced with a ring shaped circular reinforcing rib 57.

FIG. 8 is, with respect to FIG. 7, completed only by the addition of the articulation parts 50 and 52, in this example a cap 50 and the artificial glenoid 52. FIG. 9, which illustrates a schematic section parallel to the rotation axis 30 through articulation points 55, depicts a hyperbole 61. This hyperbole 61 is the cutting curve between a plane lying transversally to the pivoting axis 45 through the two contact points 55 of the circular edge 43 with the radius 63 and the lateral surface 25 shaped like a truncated cone and completed to a cone. For the lateral surfaces that do not have a conical shape the cutting curve results in a different geometrical curve.

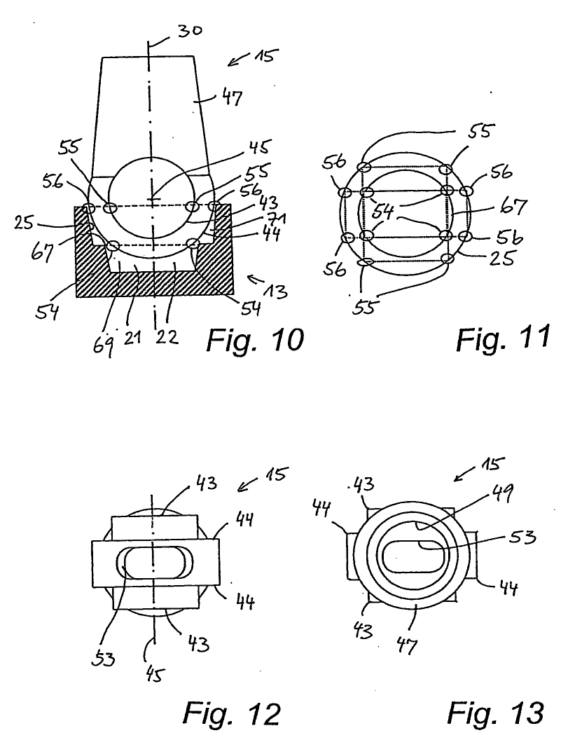

The second exemplary embodiment depicted in FIGS. 10 to 13 shows a terraced articulation cavity 22 and an articulation body 15 with two sets of two circular edges 43 and 44. Because of the gradation of the articulation cavity 22 there is a ring tip 67 in the articulation cavity at the margin of the inner and smaller hole 69. The circular edges 43 only interact with the lateral surface 25 of the larger hole 71. With four contact points 55 (which highlight smaller ellipses) between the circular edge 43 and the lateral surface 25 a clamping action is obtained by pushing the articulation body 15 into the articulation cavity 22, as described in the first embodiment example.

The circular edges 44 however have a radius that is larger than the one of the circular edges 43 and are arranged at a shorter distance from each other than the circular edges 43. The circular edges 44 interact both with the lateral surface 25 and the ring edge 67. At the contact points 56 between the circular edge 44 and the lateral surface 25 the angle between the lateral surface and the rotation axis 30 is small.

At the contact points 54 between the ring edge 67 and the circular edge 44, an cutting curve between the rotation surface, formed by the rotation of the conical sheath line 95 around the rotation axis 30 according to FIG. 14, and a plane containing the circular edge 44, has a vertex radius which is smaller than the radius of the circular edge 44. The angle between this rotation surface and the rotation axis at contact points 54 is essentially greater than the angle between the lateral surface 25 and the rotation axis 30. By this, with the eight contact points 55, 56, a clamping action is obtained at the lateral surface 25, while the four contact points 54 limit the insertion depth in ring edge 67.

In the schematic representation of the first and second exemplary embodiments, the circular edges 43,44 are represented as edges of cylindrical bodies. These circular edges can also be formed on bodies of other shapes.

FIG. 11 shows two circles. These circles represent the cutting circles in the planes of the respective contact points 54,55,56. The contact points form the angular points of three rectangles. A rectangle is formed by the contact points 54 between the ring edge 67 and the circular edge 44. A second rectangle is formed by the contact points 55 between the lateral surface 25 and the circular edges 43. A third rectangle is formed by contact points 56 between the lateral surface 25 and the circular edges 44. By means of these twelve contact points a stable support of the articulation body 15 in the articulation cavity 22 can be ensured.

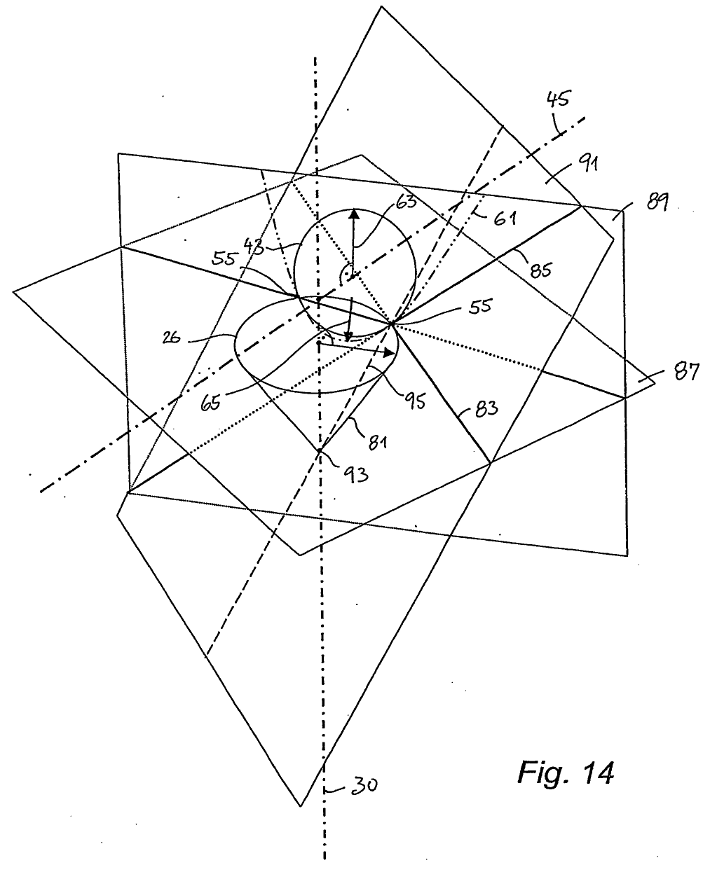

As in the first example (FIG. 1 to 8) there is also in the second embodiment (FIG. 10 to 13) a cone 47 formed at the articulation body 15 to receive an articulation part. This cone can be pivoted around the pivoting axis 45 and can be rotated around the rotation axis 30. For the fixing of the articulation body 15 in the articulation cavity, the articulation body 15 has as well a central hole 49 in the cone 47, that has a spherical bottom and a slotted hole 53 (compare FIGS. 12 and 13). Through the slotted hole 53 a screw can be connected to the head section 13 of the base part 11. To secure the screw clamping adequate tools can be used such as pressure lids, counter-screws, deformable inserts etc. As locking means a union nut can also be used to be inserted into the thread of the outer side of the head section 13 thereby pushing the circular edge 43 and the articulation body 15 into the articulation cavity 21.

FIG. 14 illustrates an exemplary geometry. This geometry ensures that the articulation body has contact with the lateral surface 25 on two contacts points 55 by a circular edge 43 having the radius 63. The contact points 55 of a circular edge 43 lie on circular line 26 around a rotation axis 30. The circular line 26 is the intersecting line of a plane 87 running transversally to the rotation axis 30 through the lateral surface 25 of the conical sheath 81 and therefore it is part of the lateral surface 25. Regardless of the shape of the lateral surface 25, this circular line 26 can be fundamental for the clamping between the articulation body and the articulation cavity. Put simply, the cone angle determining the clamping action is half of the intersecting angle of the represented conical sheath 81. In case of a conical lateral surface 25, this conical sheath 81 coincides with the lateral surface. However, this does not occur in all other cases. This conical sheath 81 can be constructed in the following way: two tangents 83,85 are laid at contact point 55. The tangent 83 lies on a plane 87 normal to the rotation axis 30, while the other tangent 85 lies at a plane 89 normal to the pivoting axis 45. Both tangents 83,85 define a tangential plane 91 at the contact point 55. This tangential plane 91 intersects the rotation axis 30 at the cone tip 93. A rotation of the conical sheath line 95 going through the cone tip 93 and the contact point 55 around the rotation axis 30 produces the conical sheath 81. The cutting curve 61 between the conical sheath 81 and the plane 89 normal to the pivoting axis 45 is a hyperbole with a vertex radius 65 smaller than the radius 63 of the circular edge 43.

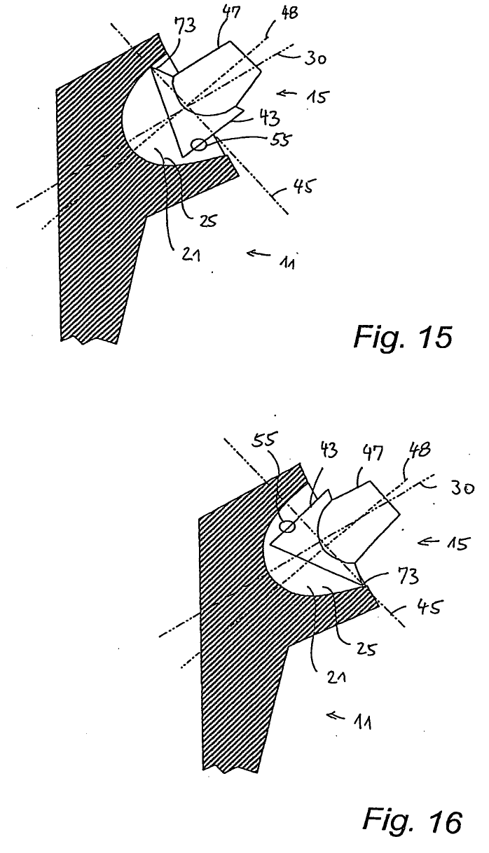

This geometry can be implemented by one single circular edge 43 only. FIGS. 15 and 16 show an exemplary embodiment with one single circular edge 43 and one tip 73. The three point structure obtained in this way, as well as a four point structure, enables the pivoting of the articulation body 15 around a third axis normal to the pivoting axis 45 and the rotation axis 30. The pivoting around this third axis results in a displacement of the articulation body in the direction of the rotation axis 30. This offset is clearly shown in both representations in FIGS. 15 and 16. Both articulation bodies 15 are pivoted around the third axis with the same angle. Depending on whether the tip 73 is raised or lowered, a different position of the collar axis 48 with regards to rotation axis 30 is obtained.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

1. An endoprosthesis for replacing a joint, comprising:
a base part capable of being anchored to a bone;
an articulation cavity arranged in the base part, tapering to the bottom of the cavity and having a rotationally symmetrical lateral surface;
an articulation body in the articulation cavity, which articulation body is supporting a first articulation part for articulation with a second, natural or artificial articulation part and has an articulation surface that interacts with the lateral surface of the articulation cavity at contact points, which articulation surface is at each contact point arranged rotationally symmetrical with respect to a pivoting axis intersecting the rotational axis; and
locking means for fixing the articulation body in the articulation cavity, wherein the articulation surface interacts with the lateral surface at at least three separate contact points, wherein:
the articulation surface comprises at least one rotationally symmetric circular edge about the pivoting axis, interacting with the lateral surface at two separated contact points,
and one of these contact points of the circular edge is located on the one side of a plane containing the pivoting axis and the rotational axis while the other contact point of the circular edge is located on the other side of this plane.

2. The endoprosthesis in accordance with claim 1, wherein the radius of the circular edge is greater than the radius of a vertex circle of an cutting curve between a plane through the contact points of the circular edge lying vertically with respect to the pivoting axis and a conical sheath formed by the rotation about the rotational axis of a straight line intersecting the rotational axis and the contact point, with this straight line belonging to a tangential plane defined by a first tangent and a second tangent at the contact point, with the first tangent running tangent to the circular edge in the plane lying perpendicularly with respect to the pivoting axis and with the second tangent running tangent to the lateral surface in the plane lying perpendicularly with respect to the rotational axis.

3. The endoprosthesis in accordance with claim 1, wherein the lateral surface exhibits a conical structure with straight lateral surface lines or trumpet-shaped lateral surface lines curved convexly towards the rotational axis or egg cup-shaped lateral surface lines curved concavely towards the rotational axis.

4. The endoprosthesis in accordance with claim 1, wherein the lateral surface exhibits at least one annular edge.

5. The endoprosthesis in accordance with claim 2, wherein the angle of the conical sheath with respect to the rotational axis is within a range in which clamping action between the lateral surface and the articulation surface can be achieved, preferably greater than 0° and less than 30°, preferentially less than 15°.

6. The endoprosthesis in accordance with claim 1, wherein the articulation body exhibits a central hole with a rotationally symmetric base, the rotation center or the rotational axis of the rotationally symmetric base more or less coincides with the pivoting axis, and an opening is formed in the base of the articulation body, and a locking means is provided for connecting the articulation body and the base part through the opening.

7. The endoprosthesis in accordance with claim 6, wherein the opening is designed as a slot with its longitudinal extension running in the swivel direction about the pivoting axis.

8. The endoprosthesis in accordance with claim 1, wherein the locking means features a threaded screw arranged axially with respect to the rotational axis.

9. The endoprosthesis in accordance with claim 1, wherein the locking means features one out of a screw cap, a contact plate and a union nut.

10. The endoprosthesis in accordance with claim 1, wherein the circular edge is square-edged.

11. The endoprosthesis in accordance with claim 1, wherein the circular edge is formed as a rounded structure in a radial section.

12. The endoprosthesis in accordance with claim 1, wherein the circular edge is interrupted and exhibits in particular peaks or notches.

13. The endoprosthesis in accordance with claim 12, wherein the lateral surface is grooved in the direction of the circumference.

14. The endoprosthesis in accordance with claim 12, wherein the lateral surface is grooved in the direction of the lateral surface lines.

15. The endoprosthesis in accordance with claim 12, wherein the lateral surface exhibits a grid relief.

16. The endoprosthesis in accordance with claim 13, wherein the dimensions of the peaks or notches of the circular edges and the dimensions of the grooves match each other such that they engage in or mesh with each other.

17. The endoprosthesis in accordance with claim 14, wherein the dimensions of the peaks or notches of the circular edges and the dimensions of the grooves match each other such that they engage in or mesh with each other.

18. The endoprosthesis in accordance with claim 15, wherein the dimensions of the peaks or notches of the circular edges and the dimensions of the grid relief match each other such that they engage in or mesh with each other.

19. The endoprosthesis in accordance with claim 1, wherein the lateral surface is harder than the circular edge.

20. The endoprosthesis in accordance with claim 1, wherein the lateral surface is softer than the circular edge.

21. The endoprosthesis in accordance with claim 1, wherein an edge of an opening in the articulation cavity exhibits a reinforcement rib on the circumference of the edge of the opening.

22. (canceled)

23. The endoprosthesis according to claim 30, wherein the articulation body is pivotably disposed in the articulation cavity for locking with the locking screw.

24. The endoprosthesis according to claim 30, wherein the articulation surface interacts with the conical lateral surface of the articulation cavity at at least three separate contact points, of which a first contact point is located on the one side of a plane containing the rotational axis and a second of these contact points, while a third contact point of the articulation surface is located on the other side of this plane, the articulation surface being rotationally symmetrical with respect to a pivoting axis intersecting the rotational axis at each contact point.

25. The endoprosthesis according to claims 24, wherein at the contact points the angle between the conical lateral surface and the rotational axis is within a range in which clamping action between the lateral surface and the articulation surface can be achieved.

26. An endoprosthesis in accordance with claim 1, wherein the joint is a shoulder joint.

27-29. (canceled)

30. Endoprosthesis for replacing a shoulder joint, comprising:
a base part capable of being anchored to a humerus bone;
an articulation cavity arranged in the base part, tapering to the bottom of the cavity and having a conical lateral surface symmetrical to a rotational axis, the angle between the conical lateral surface and the rotational axis being within a range between 0° and 15°;
an articulation body in the articulation cavity, which has an articulation surface that interacts with the conical lateral surface of the articulation cavity, and is fixable in the articulation cavity in any position desired in term of inclination and rotation, and supports a first articulation part for articulation with one out of a natural and an artificial glenoid; and
a locking screw arranged axially with respect to the rotational axis of the articulation cavity for locking the articulation body in the articulation cavity.

31. Endoprosthesis for replacing a shoulder joint, comprising:
a base part capable of being anchored to a humerus bone;
an articulation cavity arranged in the base part, tapering to the bottom of the cavity and having a conical lateral surface symmetrical to a rotational axis, the angle between the conical lateral surface and the rotational axis being within a range between 0° and 15°;
an articulation body in the articulation cavity, which has an articulation surface that interacts with the conical lateral surface of the articulation cavity, and is fixable in the articulation cavity in any position desired in term of inclination and rotation, and supports a first articulation part for articulation with one out of a natural and an artificial glenoid; and
a locking screw arranged axially with respect to the rotational axis of the articulation cavity for locking the articulation body in the articulation cavity, the articulation body having a central bore with a base, an opening formed in the base, and the locking screw connecting the articulation body and the base part through the opening.
